(19) United States
(12) Patent Application Publication
(10) Pub. No.: US 2026/0090821 A1
(43) Pub. Date: Apr. 2, 2026

(54) INTRAVASCULAR CATHETER HAVING AN EXPANDABLE PORTION

(71) Applicant: VentureMed Group, Inc., Plymouth, MN (US)

(72) Inventors: John P. Pigott, Sylvania, OH (US); Jenny Zeroni, Plymouth, MN (US); Adam Tschida, Brooklyn Park, MN (US)

(21) Appl. No.: 19/352,066

(22) Filed: Oct. 7, 2025

Related U.S. Application Data
(63) Continuation-in-part of application No. 17/856,435, filed on Jul. 1, 2022, now Pat. No. 12,433,633.
Application No. 17/856,435 is a division of application No. 17/221,539, filed on Apr. 2, 2021, now Pat. No. 12,324,603.

(51) Int. Cl. A61B 17/3207; A61B 17/00; A61B 17/3209

(52) U.S. Cl. CPC A61B 17/320725; A61B 17/3209; A61B 2017/00986; A61B 17/32075

(57) ABSTRACT

An intravascular catheter device includes an expandable portion having a proximal common end secured to a distal end of a flexible catheter tube, a distal common end spaced apart from the same, and struts extending therebetween. The expandable portion is controllably moved between a first position where at least a mid-portion of each of the struts is moved outward so as to extend beyond an outer diameter of the catheter tube and a second position where the mid-portion of the struts are moved inward so that the struts extend at or within an outer diameter of the catheter tube. A tip member is located distal to a distal end of the expandable portion, and a limiter is interposed between a distal end of the expandable portion and a proximal end of the tip member.

### CROSS-REFERENCE TO RELATED APPLICATIONS

This application is a continuation-in-part of US Application No. Ser. No. 17/856,435 filed Jul. 1, 2022, which is a divisional of U.S. application Ser. No. 17/221,539 filed Apr. 2, 2021, now U.S. Pat. No. 12,324,603 issued Jun. 10, 2025, the disclosures of each of the foregoing being hereby incorporated herein by reference as if fully restated herein.

### BACKGROUND AND SUMMARY OF THE INVENTION

This invention relates in general to intravascular catheters, such as can be used during minimally invasive surgical procedures. In particular, this invention relates to an intravascular catheter having an expandable portion, such as with incising elements.

Atherosclerosis is a chronic condition in which atheromatous plaque accumulates on the inner walls of a blood vessel. As a result, the blood vessel walls can become inflamed and, over time, may harden to form atherosclerotic lesions that cause a narrowing of the vessel lumen. In severe cases, the atherosclerotic lesions can rupture and induce the formation of thrombus (i.e., blood clots), which can prevent blood flow through the narrowed vessel lumen.

There are known procedures and devices for treating or otherwise reducing the risks associated with atherosclerosis. For example, an angioplasty is a procedure in which a balloon catheter is inserted into a narrowed region of the vessel lumen via a delivery catheter. The balloon catheter includes a flexible tube having an inflatable balloon at an end thereof. Once positioned in the narrowed region, the balloon is inflated in order to dilate the narrowed vessel lumen. The pressure in the balloon is generally sufficient to compress the accumulated plaque. However, in some cases it would be desirable to fragment the atherosclerotic lesions. Thus, it would be desirable to provide an intravascular catheter having an expandable portion that can be selectively controlled by a user and adapted to create incisions in atherosclerotic material to facilitate fragmentation of the material during an angioplasty procedure.

This invention relates to an intravascular catheter device for use during a surgical procedure. The catheter device includes a catheter tube having an expandable portion with a plurality of struts each defining an outer surface. The expandable portion is operable between a closed position, wherein the expandable portion has a first diameter, and an opened position, wherein the expandable portion has a second diameter that is larger than the first diameter. An incising element is provided on the outer surface of at least one of the struts. The incising element has a sharpened edge that extends outwardly in a radial direction from the outer surface of the strut for creating an incision in atherosclerotic material located within a blood vessel when the expandable portion is in the opened position.

Various aspects of this invention will become apparent to those skilled in the art from the following detailed description of the preferred embodiments, when read in light of the accompanying drawings.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is a plan view of a catheter device that includes a handle assembly and a catheter tube having an expandable incising portion, in accordance with a first embodiment of this invention.

FIG. 2 is a cross-sectional side view of the handle assembly taken along section line 2-2 shown in FIG. 1 when the catheter device is in a first operating mode.

FIG. 3 is an enlarged cross-sectional side view of the catheter tube taken along section line 3-3 shown in FIG. 1 illustrating the expandable incising portion disposed within a blood vessel.

FIG. 4 is a cross-sectional end view of the expandable incising portion taken along section line 4-4 shown in FIG. 3.

FIG. 5 is a cross-sectional side view of the handle assembly taken along section line 2-2 shown in FIG. 1 when the catheter device is in a second operating mode.

FIG. 6 is an enlarged cross-sectional side view of the catheter tube taken along section line 3-3 shown in FIG. 1 illustrating the expandable incising portion in an opened position.

FIG. 7 is a cross-sectional end view of the expandable incising portion taken along section line 7-7 shown in FIG. 6.

FIG. 8 is an enlarged side view of a catheter tube having an expandable incising portion, in accordance with a second embodiment of this invention.

FIG. 9 is a side view of the catheter tube shown in FIG. 8 illustrating the expandable incising portion in an opened position.

FIG. 10 is a cross-sectional end view of the expandable incising portion taken along section line 10-10 shown in FIG. 9.

FIG. 11 is an enlarged side view of a catheter tube having an expandable incising portion, in accordance with a third embodiment of this invention.

FIG. 12 is a side view of the catheter tube shown in FIG. 11 illustrating the expandable incising portion in an opened position.

FIG. 13 is an end view of the catheter tube as shown in FIG. 12.

FIG. 14 is an enlarged side view of a catheter tube having an expandable incising portion, in accordance with a fourth embodiment of this invention.

FIG. 15 is a side view of the catheter tube shown in FIG. 14 illustrating the expandable incising portion in an opened position.

FIG. 16 is an end view of the catheter tube as shown in FIG. 15.

FIG. 17 is a perspective view of an exemplary portion of an exemplary handle assembly for use with the device of FIGS. 1-16.

FIG. 18 is a perspective view of another exemplary embodiment of the expandable incising portion.

FIG. 19 is a plan view of an exemplary expandable incising portion partially assembled.

FIG. 20 is a side sectional view of another exemplary embodiment of the handle assembly of the catheter device.

FIG. 21 is a perspective view of another exemplary embodiment of the expandable portion of the catheter device.

FIG. 22 is a front sectional view of the catheter device taken along section line A-A of FIG. 21.

### DETAILED DESCRIPTION OF THE PREFERRED EMBODIMENTS

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

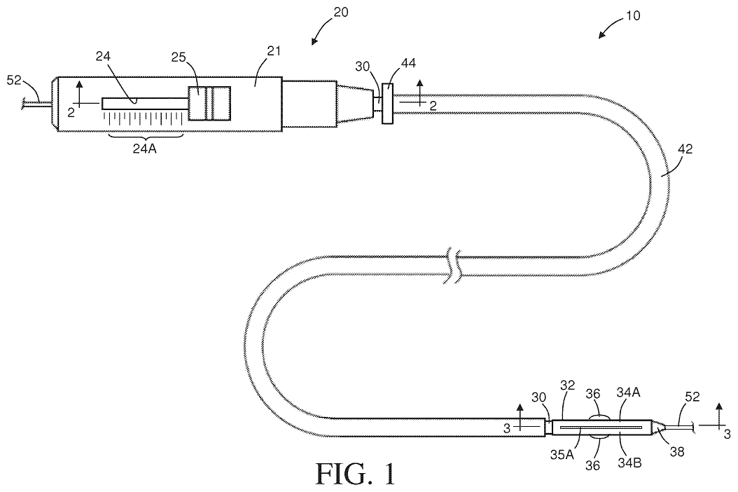

Referring now to the drawings, there is illustrated in FIG. 1 a catheter device, indicated generally at 10, in accordance with this invention. The illustrated catheter device 10 is configured to treat or reduce the risks associated with atherosclerosis. In general, the catheter device 10 includes an expandable incising portion that can be inserted into a blood vessel and expanded to create incisions in atherosclerotic material that has accumulated on inner walls of the blood vessel. The incisions facilitate the fragmentation of the atherosclerotic material during a subsequent angioplasty or atherectomy procedure. Although the catheter device 10 will be described and illustrated in the context of treating atherosclerosis, it should be appreciated that the catheter device 10 can be used in any desired environment and for any desired purpose.

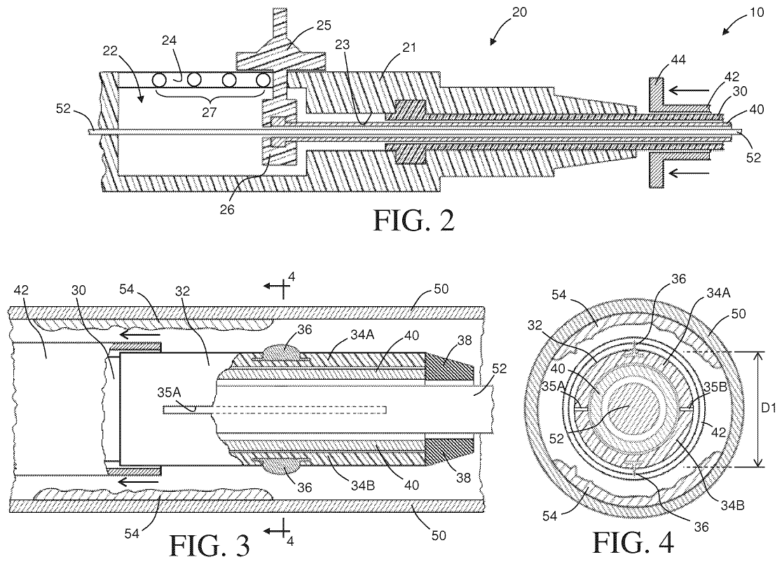

Referring now to FIGS. 1 and 2, the illustrated catheter device 10 includes a handle assembly, indicated generally at 20. The illustrated handle assembly 20 includes an elongated, cylindrical handle body 21. The handle body 21 may alternatively have any other shape that is suitable for easy handling by a surgeon. Further, the handle body 21 can be made from any suitably rigid material including, but not limited to, stainless steel or polymers.

As shown in FIG. 2, the illustrated handle body 21 defines an internal chamber 22. A passage 23 extends into an end portion of the handle body 21 for communication with the internal chamber 22. The handle body 21 further includes a slot 24 that extends through a side wall thereof for communication with the internal chamber 22. The illustrated slot 24 may have any length or width as desired. As shown in FIG. 1, an indicator 24A may be provided on the handle body 21 adjacent to the slot 24. For example, the indicator 24A can be a visual scale or any other indicating means, the purpose of which will be explained below.

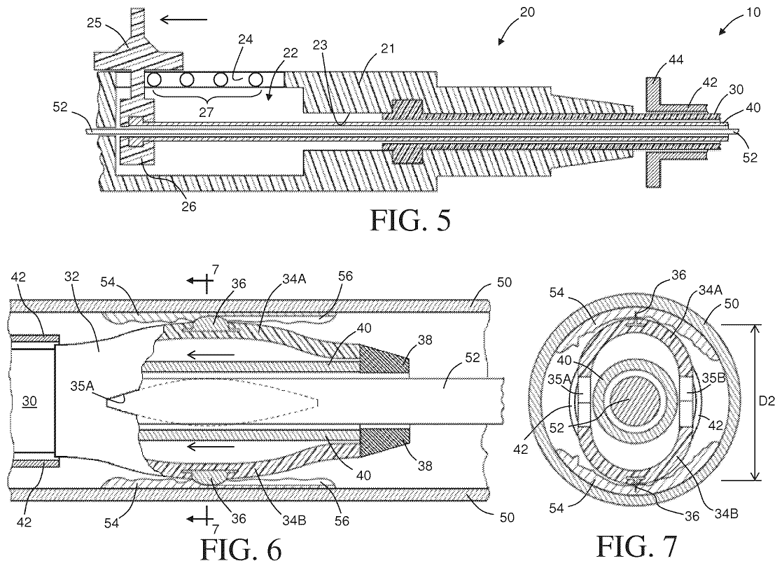

The illustrated handle assembly 20 also includes a control member 25 that is supported on the handle body 21 for sliding movement within the slot 24. For example, the control member 25 is movable between a forward position (shown in FIG. 2), a rearward position (shown in FIG. 5), or any position therebetween, which will be further explained below. As shown in FIG. 2, the illustrated control member 25 includes a base portion 26 that is disposed within the internal chamber 22 of the handle body 21. The base portion 26 may define an outer cross-sectional shape that generally corresponds with a cross-sectional shape of the internal chamber 22, although such is not required. Alternatively (or in addition), the control member 25 may be movably supported on the handle body 21 by a bearing, a bushing, a guide rail, or any other structural means. In other embodiments, the control member 25 may be supported for rotational movement, pivotal movement, or any other type of movement relative to the handle body 21, the purpose of which will become apparent below. The visual indicator 24A, described above, is configured to identify the relative position of the control member 25 with respect to the handle body 21.

The illustrated handle assembly 20 also includes a locking mechanism 27 that is configured to temporarily secure the control member 25 in a desired position, although such is not required. As shown in FIG. 2, the illustrated locking mechanism 27 includes a plurality of protrusions that are spaced apart from one another along an inner surface of the slot 24. The control member 25 frictionally engages the protrusions to hold the control member 25 in the desired position. Alternatively, the locking mechanism 27 may be a threaded fastener, a pivotal latch, a push-button release, or any other mechanism that is configured to secure the control member 25 in a desired position.

Referring now to FIGS. 1 through 3, the illustrated catheter device 10 also includes a catheter tube 30 that extends from the handle assembly 20. The catheter tube 30 is an elongated, flexible member having a proximal end that is secured to the handle assembly 20 and a distal end that extends therefrom. The catheter tube 30 can be made from any biocompatible material including, but not limited to, polyvinyl, polyethylene, nitinol, or stainless steel. Further, the catheter tube 30 can have any outer diameter, length, or wall thickness.

As shown in FIG. 2, the proximal end of the catheter tube 30 is secured to the handle body 21 and communicates with the internal cavity 22 through the passage 23. The catheter tube 30 may be secured to the handle body 21 using a flanged connection, a fused connection, an adhesive, a press-fit connection, a threaded connection, or any other securing means. Alternatively, the catheter tube 30 may be secured to the handle body 21 using a connector or any other type of attachment device.

As shown in FIGS. 1 and 3, an expandable portion 32 is provided on the distal end of the catheter tube 30. The illustrated expandable portion 32 is a cylindrical member having a longitudinal axis. The expandable portion 32 can be made from a generally resilient material that is able to flex between various positions, such as polyvinyl, polyethylene, nitinol, or stainless steel. The expandable portion 32 can be secured to the catheter tube 30 in any manner including, but not limited to, a fused connection, an adhesive, a press-fit connection, a threaded connection, or any other securing means. Alternatively, the expandable portion 32 can be integrally formed from the catheter tube 30. Further, the expandable portion 32 can have any outer diameter, length, or wall thickness.

The illustrated expandable portion 32 has a pair of struts 34A and 34B. The illustrated struts 34A and 34B are separated by a pair of longitudinally extending slits 35A and 35B that extend through side walls of the expandable portion 32. As shown in FIG. 4, the slits 35A and 35B are equally spaced apart from one another around the circumference of the expandable portion 32 such that the struts 34A and 34B have the same circumferential widths, although such is not required. The struts 34A and 34B may have any length, circumferential width, or cross-sectional shape as desired.

As shown in FIGS. 3 and 4, the illustrated expandable portion 32 also includes a pair of incising elements 36 that are respectively provided along outer surfaces of the struts 34A and 34B. The incising elements 36 can be atherotomes or other incising members having arcuate shaped sharpened edges, for example, that are configured to create incisions in atherosclerotic material as will be explained below. The illustrated incising elements 36 extend parallel with the longitudinal axis of the expandable portion 32 and outwardly in a radial direction therefrom. The incising elements 36 are equally spaced apart from one another around the circumference of the expandable portion 32. The expandable portion 32 may, however, have any number or configuration of incising elements 36 provided around the circumference thereof. Further, the incising elements 36 can have any cross-sectional shape, longitudinal length, or height and can be made from any suitable material including, but not limited to, tempered steel, stainless steel, high carbon steel, or ceramics. The incising elements 36 can be molded with the struts 34A and 34B or may otherwise be secured thereto in any manner such as, for example, using a welded or soldered connection, an adhesive, or any other fastening means.

The distal end of the expandable portion 32 may optionally include a tip member 38. The illustrated tip member 38 has a generally conical shape that facilitates insertion of the catheter tube 30 within a blood vessel 50 (see FIGS. 3 and 4) and subsequent travel therethrough. The tip member 38 may, however, have any desired shape. An aperture may axially extend through the tip member 38, the purpose of which will be explained below. The tip member 38 can be integrally formed with the expandable portion 32 or may be secured thereto, such as with an adhesive or the like. Further, the tip member 38 can be made from any biocompatible material including, but not limited to, polyvinyl, polyethylene, nitinol, stainless steel, or polyether block amide.

As shown in FIGS. 2 through 4, the illustrated catheter device 10 also includes an inner sleeve 40, although such is not required. The inner sleeve 40 is a flexible, tubular member that is supported for sliding movement within the catheter tube 30, the purpose of which will be explained below. The inner sleeve 40 can be made from any biocompatible material including, but not limited to, polyvinyl, polyethylene, nitinol, stainless steel, or a woven material. Further, the inner sleeve 40 can have any outer diameter, length, or wall thickness. The inner sleeve 40 need not be a tubular member but may alternatively be a solid wire, a braided wire, or the like.

As shown in FIG. 2, a proximal end of the inner sleeve 40 extends from the catheter tube 30 and into the internal chamber 22 of the handle body 21. The proximal end of the inner sleeve 40 is secured to the base portion 26 of the control member 25 for sliding movement therewith, the purpose of which will be explained below. The inner sleeve 40 can be secured to the base portion 26 by a flanged connection, a fused connection, an adhesive, a threaded connection, or any other securing means.

As shown in FIG. 3, the inner sleeve 40 extends through an entire length of the catheter tube 30. A distal end of the inner sleeve 40 that is opposite the handle assembly 20 is secured to the tip member 38, which is in turn secured to the expandable portion 32. The inner sleeve 40 may be secured to the tip member 38 in any manner including, but not limited to, a fused connection, an adhesive, a fastener, or the like.

Referring back to FIGS. 1 and 2, the illustrated catheter device 10 also includes a protective sheath 42 that is supported for sliding movement along an outer surface of the catheter tube 30, although such is not required. The protective sheath 42 can be made from any biocompatible material including, but not limited to, polyvinyl, polyethylene, nitinol, or stainless steel. Further, the protective sheath 42 can have any outer diameter, length, or wall thickness. The purpose of the protective sheath 42 will be explained below.

The illustrated protective sheath 42 includes a flange 44 that facilitates sliding movement of the protective sheath 42 relative to the catheter tube 30. The illustrated flange 44 is an annular member that is located at an end of the protective sheath 42 nearest the handle assembly 20. The flange 44 can be integrally formed with the protective sheath 42 or may otherwise be secured thereto in any manner, such as with an adhesive or the like. It should be appreciated that the flange 44 can have any shape or may alternatively be configured in any manner to accomplish the functions described herein and below.

The operation of the catheter device 10 will now be described with reference to FIGS. 1 through 7. Referring initially to FIGS. 1 through 4, the catheter device 10 is illustrated in a first operating mode. In the first operating mode, the control member 25 on the handle assembly 20 is located in the forward position relative to the handle body 21. The inner sleeve 40 fully extends into the catheter tube 30 such that the expandable portion 32 is in a closed position, as shown in FIGS. 3 and 4. In the closed position, the struts 34A and 34B are generally parallel with one another and with the inner sleeve 40. The slits 35A and 35B (illustrated by the dashed lines in FIG. 3) remain in a generally closed configuration. As such, the expandable portion 32 defines an initial diameter D1, which is generally the same diameter as the remaining length of the catheter tube 30. The initial diameter D1 of the expandable portion 32 may, however, be any desired dimension.

When the catheter device 10 is in the first operating mode, the distal end of the catheter tube 30 can be percutaneously inserted into a blood vessel 50, as shown in FIGS. 3 and 4. The illustrated catheter tube 30 is then advanced through the blood vessel 50 along a guide wire 52, which extends through the catheter device 10. For example, the guide wire 52 may fully extend through the inner sleeve 40, into the internal chamber 22 of the handle body 21, and exit a rear end of the handle assembly 20 (see FIG. 2). The catheter tube 30 is advanced along the guide wire 52 until the expandable portion 32 is positioned in a narrowed region of the blood vessel 50 caused by atherosclerotic material 54. Alternatively, the catheter tube 30 can be inserted into the blood vessel 50 and guided therethrough by a delivery catheter (not shown) or any other suitable procedure. During insertion and advancement of the catheter tube 30 through the blood vessel 50, the optional protective sheath 42 is preferably positioned over the expandable portion 32, thereby preventing the incising elements 36 from coming into contact with inner walls of the blood vessel 50.

Once the expandable portion 32 is positioned in the narrowed region of the blood vessel 50, the incising elements 36 can be exposed by sliding the protective sheath 42 back from the distal end of the catheter tube 30, as indicated by the direction arrows in FIG. 3. The illustrated protective sheath 42 can be moved in this manner by pulling the flange 44 towards the handle assembly 20, which is indicated by the direction arrows in FIG. 2.

Referring now to FIGS. 5 through 7, the catheter device 10 is illustrated in a second operating mode. To achieve the second operating mode, the control member 25 is moved from the forward position to the rearward position, as indicated by the direction arrow in FIG. 5. As the control member 25 is moved to the rearward position, the inner sleeve 40 is drawn within the catheter tube 30 thereby reducing the relative length of the inner sleeve 40 with respect to the catheter tube 30. The distal end of the inner sleeve 40 is attached to the tip member 38, as described above, causing the expandable portion 32 to become axially compressed between the tip member 38 and the distal end of the catheter tube 30. As a result, the struts 34A and 34B bow or expand outwardly in a generally arcuate fashion thereby defining an opened position. In the opened position, the expandable portion 32 defines a second diameter D2 that is larger than the initial diameter D1 when the expandable portion 32 is in the closed position. As shown in FIG. 6, the incising elements 36 are respectively positioned along the radially outer most surfaces of the struts 34A and 34B. Further, the outer most surfaces of the struts 34A and 34B may define a generally flat portion along a length thereof in the opened position, the purpose of which will be explained below, although such is not required. It should be appreciated that the struts 34A and 34B can have any lengths such that the expandable portion 32 can achieve a desired overall second diameter D2 in the opened position.

During operation of the catheter device 10, the second diameter D2 can be increased or decreased by selective movement of the control member 25 between the forward and rearward positions. For example, a larger second diameter D2 can be achieved by moving the control member 25 further towards the rearward position. Conversely, a smaller second diameter D2 can be achieved by moving the control member 25 further towards the forward position. The visual indicator 24A can be used to identify the instantaneous second diameter D2 of the expandable portion 32. Alternatively (or in addition), the struts 34A and 34B may be biased in the opened position so as to automatically expand outwardly to the second diameter D2 when the protective sheath 42 is slid back from the expandable portion 32. As such, sliding movement of the protective sheath 42 relative to the struts 34A and 34B can be used to selectively control the second diameter D2. In this configuration, the inner sleeve 40 and the movable components of the handle assembly 20 may not be necessary.

When the catheter device 10 is in the second operating mode, the expandable portion 32 can be pulled along the guide wire 52 through the narrowed region of the blood vessel 50. This can be accomplished by pulling on the handle assembly 20. doing so, the incising elements 36 engage the atherosclerotic material 54 and create longitudinal incisions 56 therein. As shown in FIGS. 6 and 7, the outer surface area of the arcuate shaped struts 34A and 34B, which is adjacent to the incising element 36, is configured to ride along a surface of the atherosclerotic material 54, thereby limiting the depth of the incisions 56 and preventing the incising members 36 from cutting the walls of the blood vessel 50. The expandable portion 32 can be moved any distance along the guide wire 52 to create incisions 56 having any desired length. After the incisions 56 are made in the atherosclerotic material 54, the catheter device 10 can be returned to the first operating mode (shown in FIGS. 1 through 4) by moving the control member 25 to the forward position. In doing so, the expandable portion 32 returns to the closed position. The protective sheath 42 can be slid over the expandable portion 32 and the catheter tube 30 may be removed from the blood vessel 50.

Alternatively, the catheter device 10 can be used to create additional incisions 56 in the atherosclerotic material 54. For example, after the catheter device 10 has been returned to the first operating mode, the expandable portion 32 can be relocated within the narrowed region of the blood vessel 50. The catheter tube 30 can then be rotated within the blood vessel 50 by rotating the handle assembly 20 so as to align the incising elements 36 with other portions of the atherosclerotic material 54. The previous steps can then be repeated any number of times to make multiple passes through the narrowed region of the blood vessel 50 and create additional incisions in the atherosclerotic material 54.

Thus, it should be appreciated that the illustrated catheter device 10 is advantageous in many respects. In one example, the second diameter D2 of the expandable portion 32 can be selectively controlled by operation of the handle assembly 20 or by sliding movement of the protective sheath 42. This enables the catheter device 10 to be adapted for use in blood vessels 50 of different sizes or varying diameters. In another example, the illustrated catheter device 10 can apply varying magnitudes of radial forces to the atherosclerotic material 54 by controlling the amount of force being applied to the control member 25 on the handle assembly 20. This enables the catheter device 10 to generate sufficient radial force to create incisions 56 in atherosclerotic material 54 while reducing the potential for tearing the walls of the blood vessel 50. In yet another example, the catheter device 10 can be used to make any number of passes during a single procedure to make multiple incisions 56 in atherosclerotic material 54 of varying lengths and shapes.

Referring now to FIGS. 8 through 10, there is illustrated a catheter tube 130 having an expandable portion 132, in accordance with a second embodiment of this invention. The catheter tube 130 and the expandable portion 132 may include any structural features as described and illustrated above in the previous embodiment, although such is not required. Similar features have been numbered with common reference numerals but have been increased by 100 (i.e., 110, 120, 130, etc.). It should be appreciated that similar features are structured similarly, operate similarly, and/or have the same function unless otherwise indicated by the drawings or this specification.

For example, the catheter tube 130 may extend from a handle assembly (not shown) as described above in the first embodiment. The expandable portion 132 is provided on a distal end of the catheter tube 130 and may include a tip member 138. The catheter tube 130 may also include an inner sleeve 140 and a protective sheath (not shown), which is also described above in the first embodiment.

In the illustrated embodiment, however, the expandable portion 132 includes four struts 134A, 134B, 134C, and 134D that are respectively separated by four longitudinally extending slits 135A, 135B, 135C, and 135D. The illustrated struts 134A, 134B, 134C, and 134D each include an incising element 136, although such is not required. It should be appreciated that the expandable portion 132 may have any number or configuration of struts and incising elements as desired.

As shown in FIG. 8, the illustrated expandable portion 132 further includes recessed portions 160 that respectively extend into the outer surfaces of the struts 134A, 134B, 134C, and 134D. For example, the struts 134A, 134B, 134C, and 134D can be slightly bowed inwardly toward the inner sleeve 140 when in the closed position or, alternatively, may have a reduced thickness along a central portion thereof to create the recessed portions 160. The illustrated incising elements 136 are respectively disposed within the recessed portions 160. Thus, when the catheter tube 130 is inserted into a blood vessel, as described above, the recessed portions 160 help to prevent the incising elements 136 from coming into contact with inner walls of the blood vessel. On the other hand, when the expandable portion 132 is expanded to an opened position, as explained below, the incising elements 136 become exposed from the recessed portions 160. It should be appreciated that the recessed portions 160 can eliminate or reduce the need for the protective sheath (not shown). The guide wire 152 may extend through the entire device.

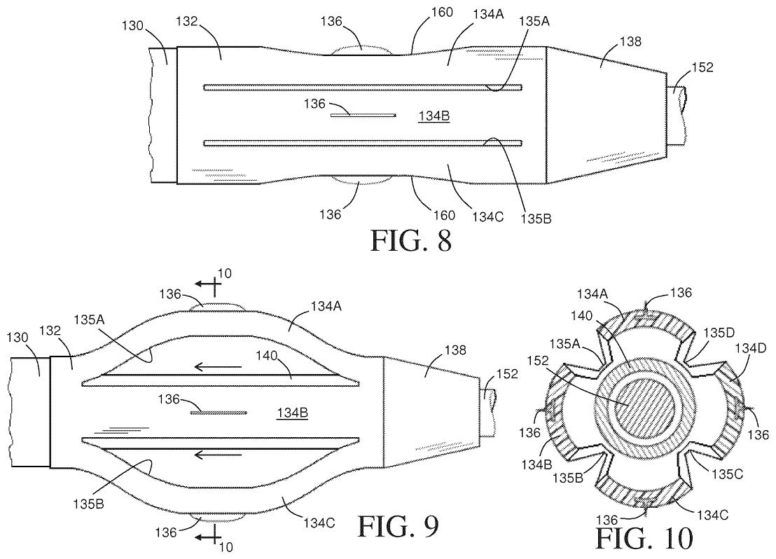

The expandable portion 132 can be operated between a closed position (shown in FIG. 8) and an opened position (shown in FIGS. 9 and 10) by selective movement of the inner sleeve 140 relative to the catheter tube 130, as described above in the first embodiment. Alternatively (or in addition), the struts 134A, 134B, 134C, and 134D can be biased in the opened position. In such an embodiment, the protective sheath (not shown) can be used to effect movement of the expandable portion 132 between the closed position and the opened position.

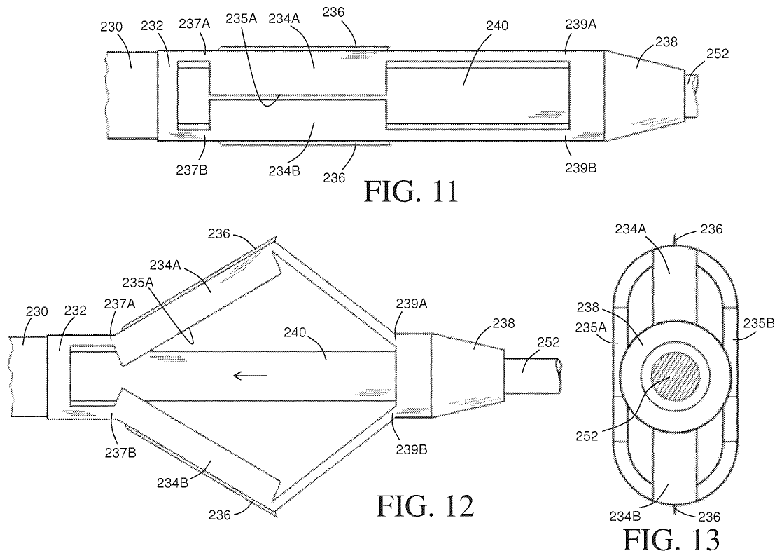

Referring now to FIGS. 11 through 13, there is illustrated a catheter tube 230 having an expandable portion 232, in accordance with a third embodiment of this invention. The catheter tube 230 and the expandable portion 232 may include any structural features as described and illustrated above in the previous embodiments, although such is not required. Similar features have been numbered with common reference numerals but have been increased by 200 (i.e., 210, 220, 230, etc.). It should be appreciated that similar features are structured similarly, operate similarly, and/or have the same function unless otherwise indicated by the drawings or this specification.

For example, the catheter tube 230 may extend from a handle assembly (not shown) as described above in the first embodiment. The expandable portion 232 is provided on a distal end of the catheter tube 230 and includes a pair of struts 234A and 234B that are separated by a pair of longitudinally extending slits 235A and 235B. The catheter tube 230 may also include a tip member 238, an inner sleeve 240, and a protective sheath (not shown), which is described above in the first embodiment. The guide wire 252 may extend through the entire device.

In the illustrated embodiment, however, the expandable portion 232 includes a first pair of weakened regions 237A, 237B and a second pair of weakened regions 239A, 239B that are respectively located at opposite ends of the struts 234A and 234B. The illustrated weakened regions 237A, 237B and 239A, 239B are formed by enlarged apertures that extend through side walls of the expandable portion 232 that function as hinges. The weakened regions 237A, 237B and 239A, 239B may help reduce the amount of bending stress in the side walls of the expandable portion 232 when the struts 234A and 234B are moved to an opened position. The struts 234A and 234B may include any number or configuration of weakened regions. Further, it should be appreciated that any of the other embodiments in this disclosure may also include weakened regions 237A, 237B and 239A, 239B.

The illustrated struts 234A and 234B remain generally flat along respective lengths thereof in both a closed position (shown in FIG. 11) and an opened position (shown in FIGS. 12 and 13) so as to form an apex, although such a configuration is not required. The incising elements 236 are provided along the generally flat portion of the respective struts 234A and 234B. As such, the incising elements 236 may also function as stiffening members for increasing the strength of the struts 234A and 234B. Further, this configuration can reduce the amount of stress in the connection between the incising elements 236 and the struts 234A and 234B, which may otherwise be caused by bowing of the struts 234A and 234B.

As shown in FIG. 12, end portions of the incising elements 236 may extend beyond the apex that is formed by each of the respective struts 234A and 234B. This configuration can increase the effective height of the incising elements 236 when the expandable portion 232 is in the opened position. As such, the incising elements 236 may have a reduced height when the expandable portion 232 is in the closed position, which may eliminate the need for the protective sheath (not shown).

The expandable portion 232 can be operated between the closed position and the opened position by selective movement of the inner sleeve 240 relative to the catheter tube 230, as described above in the first embodiment. Alternatively (or in addition), the struts 234A and 234B can be biased in the opened position. In such an embodiment, the protective sheath (not shown) can be used to effect movement of the expandable portion 232 between the closed position and the opened position.

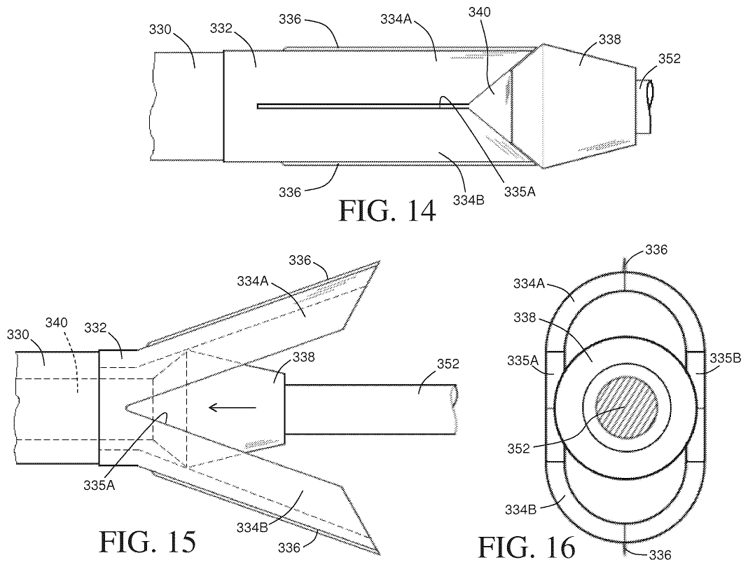

Referring now to FIGS. 14 through 16, there is illustrated a catheter tube 330 having an expandable portion 332, in accordance with a fourth embodiment of this invention. The catheter tube 330 and the expandable portion 332 may include any structural features as described and illustrated above in the previous embodiments, although such is not required. Similar features have been numbered with common reference numerals but have been increased by 300 (i.e., 310, 320, 330, etc.). It should be appreciated that similar features are structured similarly, operate similarly, and/or have the same function unless otherwise indicated by the drawings or this specification.

For example, the catheter tube 330 may extend from a handle assembly (not shown) as described above in the first embodiment. The expandable portion 332 is provided on a distal end of the catheter tube 330 and may include a tip member 338. The catheter tube 330 may also include an inner sleeve 340 that is attached to the tip member 338 and a protective sheath (not shown), which is also described above in the first embodiment. The guide wire 352 may extend through the entire device.

In the illustrated embodiment, however, the expandable portion 332 includes a pair of struts 334A and 334B that are supported thereon in a cantilevered manner (i.e., not attached to one another or to the tip member 338 at their distal ends), the purpose of which will be explained below. The struts 334A and 334B are separated by a pair of longitudinally extending slits 335A and 335B that extend from the end of the expandable portion 332. A pair of incising elements 336 is respectively provided along outer surfaces of the struts 334A and 334B. It should be appreciated, however, that the expandable portion 332 may have any number or configuration of struts and incising elements as desired.

As shown in FIGS. 15 and 16, the illustrated struts 334A and 334B are supported on the expandable portion 332 so that they can be splayed open in a Y-shaped configuration. For example, the struts 334A and 334B can be splayed open by drawing the inner sleeve 340 within the catheter tube 330, as described above in the first embodiment. In doing so, the tip member 338 slides along the inner surfaces of the struts 334A and 334B and pivots them outwardly. Alternatively (or in addition), the struts 334A and 334B can be biased in the splayed open position. In such an embodiment, the protective sheath (not shown) can be used to effect movement of the expandable portion 332 between a closed position and the splayed open position.

The struts 334A and 334B remain generally flat along their respective lengths in both a closed position (shown in FIG. 14) and the splayed open position, although such is not required. As such, the incising elements 336 may also function as stiffening members for increasing the strength of the struts 334A and 334B. Further, this configuration can reduce the amount of stress in the connection between the incising elements 336 and the struts 334A and 334B, which may otherwise be caused by bowing of the struts 334A and 334B.

As shown in FIG. 15, end portions of the incising elements 336 may extend beyond the distal ends of the respective struts 334A and 334B. This configuration can increase the effective height of the incising elements 336 when the expandable portion 332 is in the splayed open position. As such, the incising elements 336 may have a reduced height when the expandable portion 332 is in the closed position, which may eliminate the need for the protective sheath (not shown).

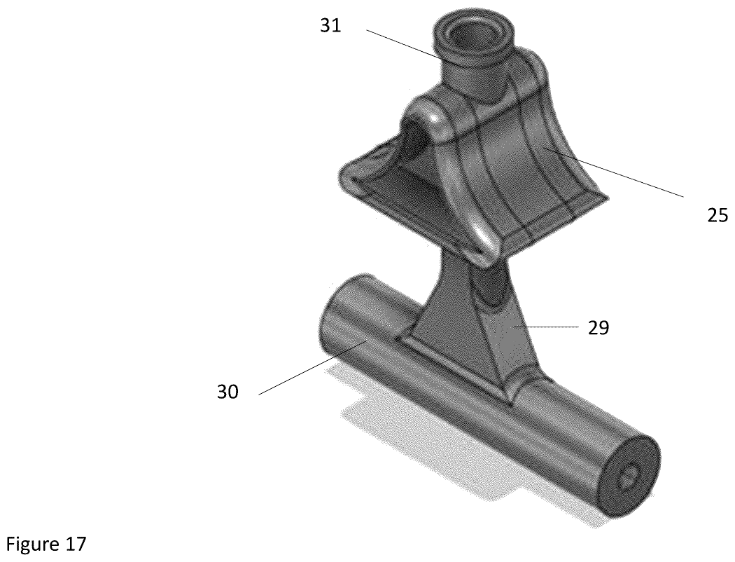

As shown in FIG. 17, the handle assembly 20 may comprise a luer lock 31. The luer lock 31 may be integrated with the control member 25 in exemplary embodiments. The control member 25 may be connected to the catheter tube 30. Such connection may be direct or indirect, such as but not limited to, the base portion 26, an intermediate element 29, combinations thereof, or the like. The luer lock 31 may be in fluid communication with an interior of the catheter tube 30. In other exemplary embodiments, the control member 25 may be connected to, directly or indirectly, the inner sleeve 40, and fluid communication between the luer lock 31 and the inner sleeve 40 may be established. For example, without limitation, one or more of said luer lock 31, said control member 25, said intermediary members 29, and said catheter tube 30 may comprise a continuous hollow passageway configured to accommodate one or more fluids, such as but not limited to, medications, saline, carbon dioxide, combinations thereof, or the like. Said hollow passageway may extend to said expandable portion 32, struts 34, and/or incising elements 36 including, for example without limitation as shown and/or described in at least U.S. Pat. No. 10,610,255 issued Apr. 7, 2020, the disclosures of which are hereby incorporated by reference as if fully restated herein. Any type or kind of luer lock 31 or other adapter for providing fluid communication from outside the device 10 to the expandable portion 32, or components thereof, may be utilized.

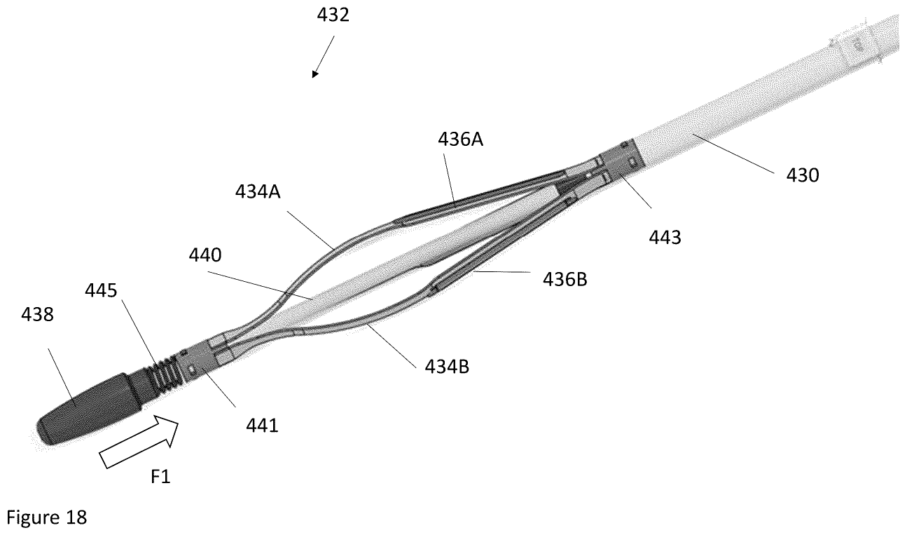

FIG. 18 illustrates another exemplary embodiment of the expandable portion 432. Similar features may be numbered with common reference numerals and increased by 100 (e.g., 10 to 110, 210, 310, or 410, etc.). The struts 434A, B, C may comprise one or more memory materials. The struts 434A, B, C may be biased in the expanded position in exemplary embodiments. For example, without limitation, the struts 434 may be heat set to expand to a particular size such that the expandable portion 432 reaches substantially a predetermined diameter when allowed to expand. In this way, when the sheath 42 is moved relative to the catheter tube 430 to expose the expandable portion 432, the struts 434 may be automatically moved into the expanded position. In such embodiments, the control member 25 may be connected to the catheter tube 430 so that movement of the control member 25, such as but not limited to advancement with the slot 24, may translate to advancement of the catheter tube 430 relative to the fixed sheath 42 so as to selectively expose the expandable portion 432 from the sheath 42 for movement into the opened position. Other movement of the control member 25, such as but not limited to retraction along the slot 24, may be configured to cause retraction of said catheter tube 430 relative to the fixed sheath 42 so as to selectively force the expandable portion 432 into the sheath 42 and into the unexpanded position.

An incising element 436A, B, C may be provided on some or all of the struts 434A, B, C. The incising elements 436 may extend along some or all of the respective one of the struts 434 on which the incising element 436 is installed, integrally formed with, mounted to, bonded to, or the like.

A first portion 443 of the expandable portion 432 may connect a proximal end of each of the struts 434 to one another and/or secure the proximal end of each of the struts 434 to the catheter tube 430, an inner sleeve 440, combinations thereof, or the like. A second portion 441 of the expandable portion 432 may connect a distal end of each of the struts 434 to one another and/or secure the distal end of each of the struts 434 to the catheter tube 430, an inner sleeve 440, tip member 438, combinations thereof, or the like. The first 443 and/or second 441 portions may be integrally formed with, bonded to, attached to, or otherwise form part of the expandable portion 432 or may be separate therefrom and connected to the same such as by adhesion, welding, fasteners, friction fit, press fit, combinations thereof, or the like.

A limiter 445 may be provide between the expandable portion 432 and the tip member 438. For example, without limitation, the limiter 445 may be provided between the second portion 441 and the tip member 438. The limiter 445 may be configured to cause the expandable portion 432 to expand only to a predetermined size. The limiter 445 may comprise one or more springs configured to provide sufficient forces F1 axially along said inner sleeve 440 and/or catheter tube 430 towards said handle assembly 20 to force said struts 434 to bow outwardly when said expandable portion 432 is exposed form the sheath 42 for expansion into the expanded position. In this way, the limiter 445 may act to bias the expandable portion 432 in the expanded position. Because the amount of force applied by the limiter 445 may be predetermined and/or limited, this may prevent the expandable portion 432 from expanding beyond a desired diameter. This arrangement may permit retraction of the sheath 42 beyond the expandable portion 432 without necessarily changing the size of the expandable portion 432 and/or the forces exerted by the incising member 436. This may be particularly advantageous where the otherwise tortuous nature of the vascular system and/or other characteristics of the access site may make precise control of the size of the expandable portion 432 by movement of the control member 25 difficult. This may also prevent over expansion of the expandable portion 432 and/or over exertion of forces at the incising elements 436.

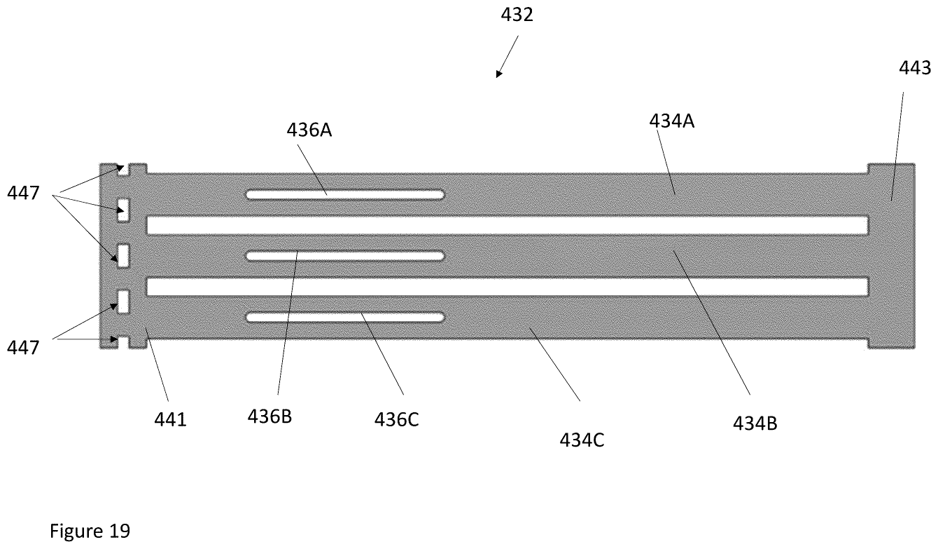

FIG. 19 illustrates a partially assembled version of the expandable portion 432. The struts 434, first portion 441, and/or second portion 443 may be formed from a common piece of material. For example, without limitation, a single piece of material, such as but not limited to, polyvinyl, polyethylene, nitinol, stainless steel, combinations thereof, or the like, may be cut or otherwise formed, then rolled, and welded to form some or all of the expandable portion 432.

In exemplary embodiments, a number of holes 447 may be formed in the second portion 441. The holes 447 may be configured for securement to corresponding portions on the catheter tube 430, the inner sleeve 440, the tip member 438, combinations thereof, or the like. Alternatively, or additionally, the holes 447 may be configured to receive portions of the limiter 445, which may also be secured to the tip member 438 and/or the inner sleeve 440. In this way, the number of welds required to create the expandable portion 432 may be reduced, such as to two welds - one on either end thereof.

The incising elements 436 may be installed on the struts 434, such as but not limited to, by adhesion, welding, press fit, combinations thereof, or the like.

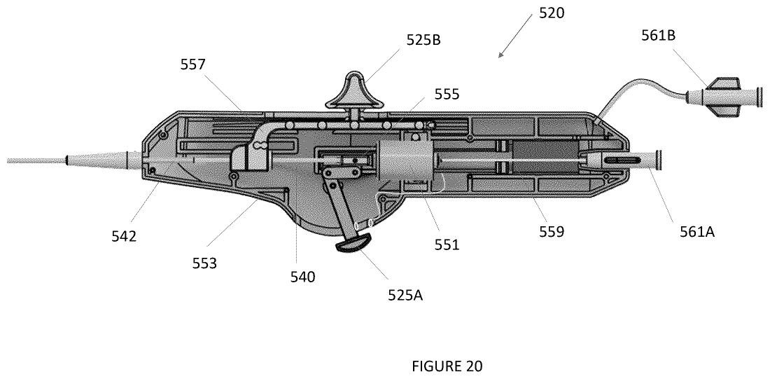

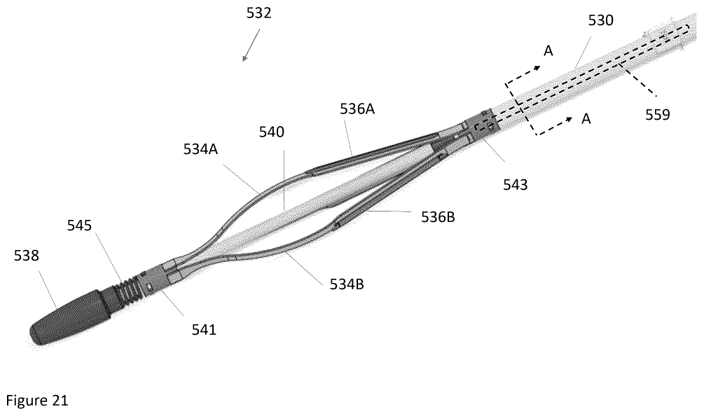

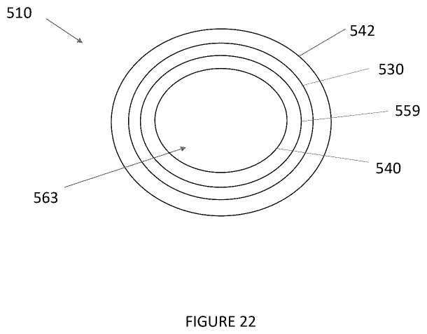

As illustrated with particular regard to FIGS. 20-22, in other exemplary embodiments, the device 510 is configured for rotational movement of the expandable portion 532, such as about a longitudinal axis of the inner sleeve 540, the catheter tube 530, and/or sheath 542. This may allow for repositioning of the struts 536 between incisions, and/or other types of tissue or other vascular material modification, such as by rotating the expandable portion 532 and/or the struts 536 thereof, to cause cutting, excision, other scoring patterns (e.g., spiral or helical shape) combinations thereof, or the like. Such rotation may be performed during axial retraction or while the expandable portion 532 is stationary.

Rotational movement of the expandable portion 532 is facilitated, at least in part, by rotational decoupling. For example, a first (proximal) and second (distal) end portion 543, 541 of the expandable portion 532 may be rotationally decoupled (and may be entirely decoupled from) from the tip member 538, inner sleeve 540, limiter 545, where utilized (the limited 545 is preferred, though optional, in such embodiments), and/or catheter tube 530. In such embodiments, movement of the expandable portion 532 between the opened/expanded and closed/collapsed positions may remain controlled by axial movement of the tip member 538 and/or limiter 545, where utilized, such as axial compression type movement of the first and second end portions 543, 541 towards one another, but where the first and second end portions 543, 541, and thereby also the struts 534, are rotationally unconstrained, thereby allowing the expandable portion 532 to move between the opened/expanded and closed/collapsed positions by axial sliding movement of the inner sleeve 540 while also permitting the struts 534 to rotate. Notably, any one or more of tip member 538, inner sleeve 540, limiter 545 may be so rotationally unconstrained to accomplish such movement. In exemplary embodiments, without limitation, the limiter 545 is connected to the second end portion 541 but disconnected from the tip member 538.

The first (proximal) and second (distal) end portion 543, 541 are preferably common to the struts 536 and extend about the inner sleeve 540. As such, they may be referred to as the first and second common end portions 532, 541 at times herein. In this regard, when the inner sleeve 540 is retracted and/or due to bias of the struts 536, the first and second common end portions 532, 541 come together and a mid-portion of the struts 536 expands outward, such as beyond an outer diameter of the catheter tube 530. When the inner sleeve 540 is extended and/or when covered by the sheath 542, the struts 536 the mid portion of the struts 536 may move inward, such as to or within the outer diameter of the catheter tube 530.

In other exemplary embodiments, the first portion 543 of the expandable portion 532 may be rotationally fixed to the catheter tube 530 which may be rotationally unconstrained and allowed to rotate with the expandable portion 532.

In exemplary embodiments, such rotational non-constraint is accomplished by way of nonattachment. For example, the tip member 538 may contact the limiter 545 but need not be connected to it for common rotational movement. Alternatively, or additionally, bushings, bearings, washers, rings, combinations thereof, or the like may be interposed between any of these components to facilitate rotational movement.

The limiter 545 (e.g., spring) may help to control movement of the expandable portion 532 and particularly the struts 534 and associated incising element 536. The interposition of the limiter 545 may regulate forces applied to the expandable portion 532 and particularly the struts 534 and associated incising elements 536, thereby providing better control. For example, the limiter 545 may absorb excess forces otherwise applied to the expandable portion 534, such as by compression, and apply additional forces where force levels drop, such as by expansion, thereby resulting in more even application of force, which may provide more uniform cutting and/or depth of incising element 536 penetration, or the like, thereby providing more consistent outcomes.

In exemplary embodiments, a rotational sleeve 559 is provided within the catheter tube 530. The rotational sleeve 559 may extend about the inner sleeve 540, which may define an inner lumen 563, such as for a guide wire. The rotational sleeve 559 may be coupled to a motor 551 at the handle assembly 520 to transfer torque and cause rotational movement of the expandable portion 532 when the motor 551 is activated. The motor 551 may be housed at least partially, if not fully, within a housing 553 of the handle assembly 520. The rotational sleeve 559 may be connected to the second end portion 543 to cause rotational movement of the expandable portion 532 with the motor. The inner sleeve 540 may extend to the tip member 538 and connect to the same for causing movement of the expandable portion between the open/expanded and closed/collapsed positions. However, the tip member 538 and limiter 545 may be rotationally decoupled from the first end portion 541 in such embodiments to facilitate the rotational movement of the expandable portion 532.

The rotational sleeve 559 may comprises a braided coil, such as in the form of braided coil reinforced tubing, though such is not required.

In other exemplary embodiments, the motor 551 is coupled to the catheter tube 530 for rotational movement. In such embodiments, the separate rotational sleeve 559 is not be required. The catheter tube 530 may comprise the braided coil to assist with torque transfer, though such is not required. In such embodiments, the catheter tube 530 may be rotationally coupled to the second end portion 543 of the expandable portion 532 to cause rotational movement of the expandable portion 532, while the first end portion 541 is rotationally decoupled form the limiter 545 and/or the tip member 538.

In other exemplary embodiments, the rotational sleeve 559 extends within the inner sleeve 540 to the tip member 538 where it is coupled to the same. In such embodiments, the tip member 538, limiter 545, and first end portion 541 are rotationally coupled to provide rotational movement of the expandable portion 532 with the motor 551. However, the second end portion 543 may be rotationally decoupled from the catheter tube 530 to facilitate such rotational movement.

In yet other exemplary embodiments, the inner sleeve 540 is coupled to the motor 551 for rotational movement. And, the inner sleeve 540 is rotational coupled to the tip member 538, which is rotationally coupled to the limiter 545 and first end portion 541 to provide the rotational movement of the expandable portion 532 with the motor 551. However, the second end portion 543 may be rotationally decoupled from the catheter tube 530 to facilitate such rotational movement. The inner sleeve 540 may comprise the braided coil to assist with torque transfer, though such is not required.

In exemplary embodiments, a first control member 525A is connected to the inner sleeve 540 such that movement of the first control member 525A moves the inner sleeve 540 and thereby the expandable portion 532. For example, the first control member 525A is a swinging lever positioned within an opening of the housing 533 for swinging movement forward and backwards, thereby causing sliding movement of the inner sleeve 540, such as by way of interposed levers. The first control member 525A is preferably located at a lower portion of the housing 533 for actuation by a user's index finger in a trigger pull type movement while gripping the handle assembly 520.

A second control member 525B may be provided at the handle assembly 520 for controlling movement of the sheath 542, where utilized. For example, the second control member 525B may comprise a slider positioned within a slot of the housing 533 for sliding movement forward and backwards, thereby causing sliding movement of a sheath 542. The second control member 525B is preferably located at an upper portion of the housing 553 for movement by a user's thumb while gripping the handle assembly 520.

Alternatively, the handle assembly 520 may be turned over for use when flipped.

The first and/or second control member 525A, 525B may optionally be biased towards a given position.

The first and second control members 525A, 525B may protrude from the housing 521 and be mechanically linked, directly or indirectly, to components inside the housing 521, such as the inner sleeve 540 and the sheath 542, respectively. The control member(s) 525A, 525B may include sliders, buttons, knobs, levers, combinations thereof, or the like. Connection may be made by way of linkages, arms, sliders, levers, springs, gears, cams, belts, combinations thereof, or the like. In exemplary embodiments, the second control member 525B is connected to a linkage arm 557 which connects to the sheath 542 but slides about the inner sleeve 540 such that the sheath 542 and inner sleeve 540 may move independently from one another.

In exemplary embodiments, a switch 555 is provided which is connected to, or within the movement path of, the first control member 525A. The switch 555 may be activated upon movement of the first control member 525A, thereby activating the motor 551 upon movement of the expandable portion 532 into the opened/expanded position. Alternatively, or additionally, the switch 555 may be located elsewhere and/or otherwise separately actuatable, such as for manual activation by an operator of the device 510 by way of separate activation device (e.g., sliders, buttons, knobs, levers, combinations thereof, or the like).

In other exemplary embodiments, a knob or other manually manipulatable object is provided in substitution for the motor 551, such as at the handle assembly 521 or otherwise external to the expandable portion 532, which engages the catheter tube 530, rotation sleeve 559, or the inner sleeve 540 as shown and/or described herein to cause rotational movement of the same, such as when the knob is rotated. For example, the knob may be provided at a proximal end of the handle assembly 520 or about the housing 553 at another portion of the handle 520.

Optionally, the struts 534 may be biased for movement into an expanded position. In such embodiments, movement of the expandable portion 532 between the open/expanded and collapsed/closed positions may be provided, at least in part, by way of the sheath 542. For example, when the sheath 542 is removed, the struts 534 may naturally move into the open/expanded position. And, when the sheath 542 is moved over the struts 534, they may be forced into the closed/collapsed position.

In such embodiments, while the inner sleeve 540 may be provided, and may be attached to the tip member 538, it need not necessarily be axially slidable. In such embodiments, the inner sleeve 540 may be coupled to the motor 551 to cause the rotational movement of the expandable portion 532, though such is not required. For example, the rotational sleeve 559 may be provide and so coupled, and/or, the struts 542 may be both biased and under control by the inner sleeve 540, for example.

Some or all of the struts 534 may include the incising element 536. In exemplary embodiments, a longitudinal axis of each of the struts 534 is at least aligned with a longitudinal axis of the underlying strut 534. The incising elements 536 may be at least substantially cuboid in shape, optionally with a sharpened edge along an upper edge thereof. This may allow incision of plaque or other material upon axial retraction of the device along a blood vessel or other vascular passageway when the expandable portion 532 is in an expanded position. In this regard, the expandable portion 532 may be placed in a collapsed/closed position and/or sheathed during rotation and unsheathed and/or placed in the opened/expanded position for repeated retraction to create additional incisions in a same or different part of a same or different vascular passageway.

However, the expandable portion 532 may be rotated when unsheathed and/or placed in the opened position to cause other types of cutting, such as during axial movement (e.g., cause spiral incisions) and/or when stationary (e.g., to excise or disrupt plaque or other material).

The incising elements 536 need not necessarily be elongationally and/or otherwise oriented or configured for axial incision. For example, without limitation, the incising elements 536 and/or sharped blades thereof may be laterally oriented, such as relative to the longitudinal axis of the struts 536. This may provide scoring of plaque or other material upon rotational movement of the expandable portion 532, such as when in the expanded positioned and/or unsheathed. This may allow for other types of cutting, such as excision for removal. Such rotational movement may be provided while the expandable portion 532 is axially stationary and/or while being axially retracted. In yet other exemplary embodiments, the incising elements 536 and/or sharped blades thereof may be oriented laterally, may be curved, scooped, combinations thereof, or the like.

While plaque and/or blood vessels are sometimes shown and/or described herein, applications are not so limited. For example, without limitation, treatment may be made to calcified lesions, fibrous build up in dialysis fistulas, cutting or otherwise disrupting synechia structures, valve cutting, combinations thereof, or the like.

While direct engagement is sometimes shown and/or described herein, such as from the motor 551 and/or the control member 252 and/or other components, those of skill in the art will recognize that indirect engagement may be provided, such as by way of intermediate shafts, gears, springs, levers, cams, belts, combinations thereof, or the like.

The handle assembly 520 may include a first port 561A, which may be located at a proximal end thereof, such as in line with the rotational sleeve 559, inner sleeve 540, and/or sheath 542. The first port 561A may be configured to accept a guide wire, such as for movement through the central lumen 563 of the device 510.

The handle assembly 520 may include a second port 561B, such as a luer lock, for receiving fluid for flushing the device 510, saline, combinations thereof, or the like. The second port 561B may be located at the proximal end of the device 510 though other locations may be utilized. The second port 561B may include a flexible connection to the device 510, such as a tube.

In other exemplary embodiments, the motor 551 is connected to the inner sleeve 540 such as to cause axial movement of the inner sleeve 540 when operated, thereby moving the expandable portion 532 between the open and closed positions. The motor 551 may be configured for rapid pulsing when activated, such as multiple times a second. This may be accomplished by way of software instructions, such as at a controller electronically connected to the motor 551. Notably, the control element 525A and/or switch 555 may be electrically and/or mechanically connected to the motor 551 and/or switch 555 for this purpose. This may cause rapid opening and closing of the expandable portion 532, which may create hydraulic waves, such as through the blood or other vascular fluid, which may crack or otherwise affect plaque, calcium, or other material provided at a zone of attention. In such embodiments, the rotation sleeve 559 is not necessarily required.

In other exemplary embodiments, the expandable portion 532, including optionally the limiter 545 and the tip member 538 may be positioned at a mid-portion of the catheter tube 530. This may allow for flexible steering of the expandable portion 532, such as along particularly sinuous vascular routes.

In such embodiments, the catheter tube 530 may be pre-angled, which may allow for effective steering through the vascular system, such as without the need for a guidewire. In such cases, the inner lumen 563 may not be required and the tip member 538 may be closed.

In other exemplary embodiments, the motor 551 may comprise and/or form part of a pump which is fluidly connected to the inner lumen 563 so as to, for example, aspirate clots or other material when activated. In such embodiments, the rotation sleeve 559 is not necessarily required and the inner lumen 563 may be provided by the inner sleeve 540.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

Certain operations described herein may be performed by one or more electronic devices. Each electronic device may comprise one or more processors, electronic storage devices, executable software instructions, combinations thereof, and the like configured to perform the operations described herein. The features and/or functionality shown and/or described herein may be accomplished by way of stored, executable software instructions, such as in the form of applications, programs, routines, combinations thereof, or the like. The electronic devices may be general purpose computers or specialized computing devices. The electronic devices may comprise personal computers, smartphones, tablets, databases, servers, or the like. The electronic connections and transmissions described herein may be accomplished by one or more wired or wireless connectivity components (e.g., routers, modems, ethernet cables, fiber optic cable, telephone cables, signal repeaters, and the like) and/or networks (e.g., internets, intranets, cellular networks, the world wide web, local area networks, and the like). The computerized hardware, software, components, systems, steps, methods, and/or processes described herein may serve to improve the speed of the computerized hardware, software, systems, steps, methods, and/or processes described herein. The electronic devices, including but not necessarily limited to the electronic storage devices, databases, controllers, or the like, may comprise and/or be configured to hold, solely non-transitory signals.

The principle and mode of operation of this invention have been explained and illustrated in its preferred embodiments. However, it must be understood that this invention may be practiced otherwise than as specifically explained and illustrated without departing from its spirit or scope.

What is claimed is:

1. An intravascular catheter device for treating a zone of attention located within a vascular passageway, said intravascular catheter device comprising:
a flexible catheter tube;
an expandable portion having a proximal common end secured to a distal end of the flexible catheter tube, a distal common end spaced apart from the proximal common end, and a plurality of struts extending between the proximal common end and the distal common end for controllable movement between a first position where at least a mid-portion of each of said plurality of struts is moved outward so as to extend beyond an outer diameter of said catheter tube and a second position where at least the mid-portion of said plurality of struts is moved inward so that the struts extend at or within the outer diameter of said catheter tube;
a tip member located distal to a distal end of said expandable portion; and
a limiter interposed between a distal end of said expandable portion and a proximal end of said tip member.

2. The intravascular catheter device of claim 1 further comprising:
an inner sleeve located within the catheter tube and connected to the tip member, wherein the inner sleeve is axially slidable within the catheter tube for moving the plurality of struts between the first position and the second position by way of imparting forces to the tip member, and thereby the limiter, and thereby the expandable portion.

3. The intravascular catheter device of claim 2 wherein:
the limiter comprises a spring extending about the inner sleeve.

4. The intravascular catheter device of claim 3 wherein:
the inner sleeve and the tip member are configured to accommodate a guide wire extending therethrough.

5. The intravascular catheter device of claim 4 wherein:
the spring is connected to the distal end of the expandable portion and is disconnected from the proximal end of the tip member.

6. The intravascular catheter device of claim 5 further comprising:
a rotational sleeve located within the catheter tube and connected to a proximal end of the expandable portion; and
a motor or knob connected to the rotational sleeve such that activation of said motor or manual rotation of the knob causes rotational movement of said expandable portion.

7. The intravascular catheter device of claim 6 wherein:
the rotational sleeve comprises braided coil reinforced tubing.

8. The intravascular catheter device of claim 6 further comprising:
a handle assembly comprising a housing and a first control member mechanically connected to the inner sleeve for causing the sliding movement of the inner sleeve, where said motor is provided within the housing of the handle assembly or said knob is provided at the housing of the handle assembly.

9. The intravascular catheter device of claim 8 further comprising:
a sheath extending over the catheter tube and axially moveable relative to the same to selectively cover the expandable portion; and
a second control member forming part of the handle assembly, wherein said second control member is mechanically connected to the sheath to cause the axial movement of the sheath.

10. The intravascular catheter device of claim 9 wherein:
the struts are biased for movement into the first position.

11. The intravascular catheter device of claim 6 further comprising:
an incising element associated with one of the plurality of struts.

12. The intravascular catheter device of claim 11 wherein:
the incising element extends laterally relative to a longitudinal axis of the associated one of the plurality of struts or is curved.

13. The intravascular catheter device of claim 1 further comprising:
an incising element associated with one of the plurality of struts.

14. The intravascular catheter device of claim 13 wherein:
the incising element extends longitudinally along a longitudinal axis of the associated one of the plurality of struts.

15. The intravascular catheter device of claim 14 wherein:
the incising element includes a sharpened edge extending along an upper edge thereof.

16. The intravascular catheter device of claim 14 wherein:
the incising element extends along a proximal half of the associated one of the plurality of struts.

17. The intravascular catheter device of claim 1 further comprising:
a motor connected to the catheter tube such that, when activated, the motor causes rotational movement of said expandable portion, and wherein the limiter and the tip member are rotationally decoupled from one another to facilitate such rotational movement.

18. The intravascular catheter device of claim 1 further comprising:
an inner sleeve located within the catheter tube and connected to the tip member, wherein the inner sleeve is axially slidable within the catheter tube for moving the plurality of struts between the first position and the second position by way of imparting forces to the tip member, and thereby the limiter, and thereby the expandable portion; and
a motor connected to the inner sleeve such that, when activated, the motor causes axial movement of the inner sleeve, and thus movement of said expandable portion between the first and second positions.

19. The intravascular catheter device of claim 1 wherein:
a portion of the catheter tube extends beyond a distal end of the tip member.

20. The intravascular catheter device of claim 1 further comprising:
an inner sleeve located within the catheter tube and connected to the tip member, wherein the inner sleeve is axially slidable within the catheter tube for moving the plurality of struts between the first position and the second position by way of imparting forces to the tip member, and thereby the limiter, and thereby the expandable portion; and
a motor providing a pump fluidly connected to an inner lumen of the inner sleeve such that, when activated, the motor causes aspiration of fluid or objects at the distal end of the inner sleeve.

21. A method for treating a zone of attention within a vascular passageway, said method comprising:
positioning the expandable portion of the intravascular catheter device of claim 1 at the zone of attention;
moving the expandable portion through the zone of attention while the expandable portion is maintained in the first position;
placing the expandable portion in the second position; and
removing the expandable portion from the zone of attention.

22. A method for treating a zone of attention within a vascular passageway, said method comprising:
positioning the expandable portion of the intravascular catheter device of claim 8 at the zone of attention;
treating the zone of attention including:
moving the expandable portion through the zone of attention while the expandable portion is maintained in the first position; and
activating the motor while the expandable portion is maintained in the first position to cause rotational movement of the expandable portion about a longitudinal axis of the inner sleeve;
placing the expandable portion in the second position; and
removing the expandable portion from the zone of attention.

23. An intravascular catheter device for treating a zone of attention located within a vascular passageway, said intravascular catheter device comprising:
a handle assembly comprising:
a housing;
a first control member; and
a second control member;
a motor provided within the housing;
a switch connected to the first control member and the motor such that movement of the first control member into a first position causes the switch to close and the motor to activate;
a flexible catheter tube extending from a distal end of the housing;
an expandable portion comprising:
a proximal common end fixed to a distal end of the flexible catheter tube;
a distal common end spaced apart from the proximal common end; and
a plurality of struts extending between the proximal common end and the distal common end for controllable movement between a first position where at least a mid-portion of each of said plurality of struts is moved outward so as to extend beyond an outer diameter of said catheter tube and a second position where at least the mid-portion of said plurality of struts is moved inward so that the struts extend at or within the outer diameter of said catheter tube; and
incising elements, each provided at an outer surface of a respective one of the plurality of struts;
a sheath extending over the catheter tube, connected to the first control element, and axially slidable relative to the catheter tube by movement of the first control element to selectively cover the expandable portion, wherein the struts are biased for movement into the first position;
a tip member located distal to, and spaced apart from, the distal comm end of said expandable portion;
an inner sleeve located within the catheter tube and connected to the tip member and the second control element such that movement of the second control element causes axial sliding movement of the inner sleeve and corresponding movement of the struts between the first and second position; and
a limiter interposed between the distal common end of said expandable portion and said tip member, the limiter comprises a spring coiled about the inner sleeve; and
a rotational sleeve comprising braided reinforced tubing located within the catheter tube and connected to the motor and the proximal common end of the expandable portion for rotating the struts when the motor is activated;
wherein the inner sleeve and the tip member are configured to accommodate a guide wire extending therethrough.
